(19) United States
(12) Patent Application Publication
(10) Pub. No.: US 2015/0216565 A1
(43) Pub. Date: Aug. 6, 2015

(54) SYSTEMS AND METHODS FOR CORRECTING A ROTATIONAL BONE DEFORMITY

(71) Applicants: Dror Paley, Laval (CA); Ariel Ricardo Dujovne, Cote St Luc (CA); Fady Rayes, Vaudreuil-Dorion (CA)

(72) Inventors: Dror Paley, Laval (CA); Ariel Ricardo Dujovne, Cote St Luc (CA); Fady Rayes, Vaudreuil-Dorion (CA)

(21) Appl. No.: 14/172,598

(22) Filed: Feb. 4, 2014

(51) Int. Cl. A61B 17/68

(52) U.S. Cl. CPC A61B 17/68

(57) ABSTRACT

Embodiments provide an orthopedic apparatus that can include a tether member that is capable of engaging a bone using a plurality of coupling members. In some aspects, the tether member can include a central band that may be disposed between a distal member and a proximal member. The proximal member can define a proximal aperture and the distal member can define a distal aperture. Moreover, the distal member can be configured to be engaged to a fixed segment of the bone using at least one of the coupling members and the proximal coupling member can be configured to be engaged to a mobile segment of the bone using at least one other coupling member.

### FIELD

This disclosure relates generally to the field of orthopedic apparatuses, and in particular, to an orthopedic apparatus and related method for correcting rotational bone deformities.

### BACKGROUND

In orthopedics, rotational deformities of the bone along the lower portions of an individual can change the planar orientation of various respective reference planes for the hip, knee, and ankle. For example, abnormal angulation of the femoral neck with respect to the transcondylar axis of the knee is referred to as femoral anteversion. In general, rotational deformities as discussed above may be defined as an abnormal angulation of a bone relative to a longitudinal axis.

It is very common in infants to be born with femoral anteversion due to the position of the fetus inside the womb during pregnancy and can occur in up to 10% of children. In fact, femoral anteversion is the most common cause of children walking with their toes inward (in-toeing) in children older than 3 years of age. Although most rotational bone abnormalities, such as femoral anteversion, are resolved under normal growth and development, a small percentage of cases will continue to suffer from a residual rotational deformity that may later require surgical correction.

One common method of surgical bone realignment to address femoral anteversion is by performing an osteotomy procedure which requires a cutting of the bone followed by realignment of the bone to the correct the bone orientation. However, osteotomy procedures require making a relatively large incision to create access to the bone for the surgeon to perform the bone cutting realignment, thereby making the procedure substantially invasive. In addition, the procedure can cause disruption of the adjacent musculature surrounding the bone as well as possibly damaging the neurovascular structures. Procedures to cut and realign bones are associated with a long and painful rehabilitation period that can last several months. The cut bone ends may not heal and in such cases, further surgery may be necessary. Implant failure is also a well-documented complication of osteotomies. Another concern is the accidental damage to the growth plate that can occur during the surgical realignment procedure, which can later inhibit healthy and normal limb growth. In addition, there are still further concerns, including the risk of infection, as well as the risk of delayed union of the bone segments, mal-union of the bone segments, and over/under correction. As such, current surgical bone realignment apparatuses and methods require a relatively invasive procedure be performed to correct rotational bone deformities.

### SUMMARY

In one embodiment, an orthopedic apparatus can include a tether member that is capable of engaging a bone using a plurality of coupling members. In some aspects, the tether member can include a central band that may be disposed between a distal member and a proximal member. The proximal member can define a proximal aperture and the distal member can define a distal aperture. Moreover, the distal member can be configured to be engaged to a mobile segment of the bone using at least one of the coupling members and the proximal coupling member can be configured to be engaged to a fixed segment of the bone using at least one other coupling member.

Some embodiments provide a method of correcting rotation deformities of a bone. The bone may include a fixed segment separated from a mobile segment by a growth plate and a first side of the bone laterally opposing a second side of the bone. The method may include coupling a first portion of a first tether member to the fixed segment of bone on the first side of the bone using a first coupling member and coupling a second portion of the first tether member to the mobile segment of the bone on the first side of the bone using a second coupling member. Moreover, the first tether member can be coupled to the fixed segment of the bone and the mobile segment of the bone such that a central band of the first tether member can be under tension and at a relative angle to an anatomical axis of the bone. The method may also include coupling a first portion of a second tether member to the fixed segment of bone on the second side of the bone using a third coupling member and coupling a second portion of the second tether member to the mobile segment of the bone on the second side of the bone using a fourth coupling member. Moreover, the second tether member can be coupled to the fixed segment of the bone and the mobile segment of the bone such that a central band of the second tether member can be under tension and at a relative angle to the anatomical axis of the bone, wherein the tension associated with the central band of the first tether member and the second tether member at the relative angle to the anatomical axis of the bone promotes torsional growth of the bone. In addition, the first tether member and the second tether member are coupled to the bone such that as growth occurs at the growth plate, the angle relative to the anatomical axis becomes closer to zero degrees.

Some embodiments provide a method of correcting rotation deformities of a bone. The bone may include a fixed segment separated from a mobile segment by a growth plate and a first side of the bone laterally opposing a second side of the bone. The method may include positioning a first coupling member through the fixed segment of the bone such that a first end and a second end of the first coupling member extend from the first side of the bone and the second side of the bone, respectively. The method may also include positioning a second coupling member through the mobile segment of the bone such that a first end and a second end of the second coupling member extend from the first side of the bone and the second side of the bone, respectively. The method may also include positioning a first tether member on the first side of the bone such that the first end of the first coupling member and the first end of the second coupling member can engage the first tether member. In addition, the method may further provide positioning a second tether member on the second side of the bone such that the second end of the second coupling member and the second end of the second coupling member can engage the second tether member, wherein the bone comprises an anatomical axis, and wherein the first tether member and the second tether member are positioned on the first side and second side of the bone at an angle relative to the anatomical axis. In addition, the first tether member and the second tether member are positioned on the first side and second side of the bone such that as growth occurs at the growth plate, wherein the angle relative to the anatomical axis becomes closer to zero degrees.

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is a perspective view of a first embodiment of an orthopedic apparatus coupled to a bone;

FIG. 2 is a perspective view of a first embodiment of a tether member;

FIG. 3 is a perspective view of a first embodiment of a coupling member;

FIG. 4 is a perspective view of a second embodiment of a tether member;

FIG. 5A is a front view of a third embodiment of a tether member;

FIG. 5B is a front view of the first embodiment of the tether member;

FIG. 6A is a perspective view of a fourth embodiment of a tether member;

FIG. 6B is a perspective view of the tether member of FIG. 6A showing a plurality of coupling members;

FIG. 7A is a perspective view of the orthopedic apparatus coupled to bone immediately after coupling;

FIG. 7B is a perspective view of the orthopedic apparatus coupled to the bone of FIG. 7A after 12 months of treatment;

FIG. 7C is a simplified graphic illustrating the relationship of length of the orthopedic apparatus, positioning angle of the orthopedic apparatus, and growth over time of the bone being treated with the orthopedic apparatus;

FIG. 8A is a perspective view of the orthopedic apparatus coupled to bone immediately after coupling;

FIG. 8B is a perspective view of the orthopedic apparatus coupled to the bone of FIG. 8A after treatment;

FIG. 9A is an anterior view of a bone that illustrates a femur condylar distance;

FIG. 9B is a distal view of a bone that illustrates multiple condylar distances;

FIG. 10A is an anterior view of a second embodiment of an orthopedic apparatus;

FIG. 10B is a lateral view of the orthopedic apparatus of FIG. 10A;

FIG. 10C is a distal view of the orthopedic apparatus of FIG. 10A;

FIG. 11 is a perspective view of a second embodiment of a coupling member;

FIG. 12 is a perspective view of a securing member; and

FIG. 13 is a perspective view of a different configuration of a securing member.

Corresponding reference characters indicate corresponding elements among the view of the drawings. The headings used in the figures should not be interpreted to limit the scope of the claims.

### DETAILED DESCRIPTION

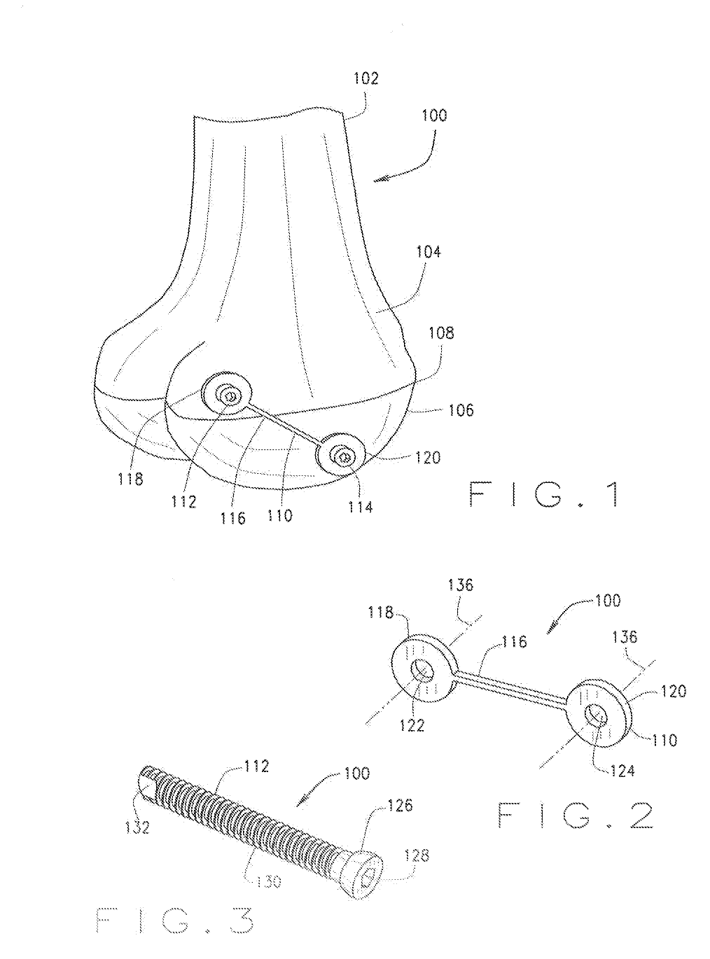

Referring to the drawings, embodiments of an orthopedic apparatus are illustrated and generally indicated as 100 in FIGS. 1-3 and 7-8. In general, the orthopedic apparatus 100 can be used to correct, repair, or otherwise improve bone maladies (e.g., bone rotational deformities) in an animal, which includes, but is not limited to humans. For example, some embodiments of the orthopedic apparatus 100 can be used to correct rotational bone deformities that are associated with any bone that has a growth plate, such as a femur, a tibia, a humerus, etc. Moreover, the following disclosure largely details the use of embodiments of the orthopedic apparatus 100 in the context of the correction of a femoral rotational bone deformity. However, this disclosure is not intended to limit the use of the orthopedic apparatus 100 to the context of correcting femur-based deformities. Rather, as mentioned above, the orthopedic apparatus 100 can be used to correct the bone maladies associated with any suitable bone in the body of an animal.

Referring to FIG. 1, in some embodiments, the orthopedic apparatus 100 can be used to correct the rotational deformities of a bone 102 (e.g., a femur, as shown in FIG. 1). In particular, the bone 102 includes a mobile segment 104 and a fixed segment 106, with a growth plate 108, which is also known as a physis, disposed between the mobile and fixed segments 104, 106. In general, growth of mammalian long bones occurs at the growth plate 108, which is complex in its anatomy and function. In the physiological context, cells in the growth plate 108 are capable of division in response to hormonal, chemical and mechanical influences. Although the exact mechanisms by which these growth processes occur are yet to be fully understood, growth occurs through continued division of cartilage cells in the growth plate 108, which is subsequently converted into bone. Growth plates 108 have the ability to lengthen the bone along an axis that bears a fixed relationship to known anatomical and mechanical axes of limbs. Although there are minor variations between individuals, the general shape of normal long bones is the result of the ability of growth plates 108 to lay down new bone along a predetermined axis along the coronal, sagittal and axial planes.

As shown in FIG. 1, in some embodiments the orthopedic apparatus 100 includes at least one tether member 110, a first coupling member 112, and a second coupling member 114. Referring now to FIGS. 1 and 2, the tether member 110 can include a central band 116, a proximal member 118, and distal member 120. In some aspects, the tether member 110 can be formed such that the central band 116, the proximal member 118, and the distal member 120 are substantially or completely integral with each other (e.g., the tether member 110 is formed with the constituent elements provided in place, using fabrication processes such as molding or casting the entire tether member 110 at one time). In other aspects, one or both of the proximal and distal members 118, 120 can be coupled to the central band 116 at any point after fabrication.

Moreover, in some aspects, the tether member 110 can be fabricated from any material that exhibits some level of flexibility to follow and/or accommodate unique bone geometries and contours. For example, the tether member 110 can be fabricated from a metal, a plastic, a polymer, an elastomer, any other suitable materials, or any combination thereof. In addition, depending on the intended use of the tether member 110, the length, width, or thickness of the central band 116 can be varied.

As illustrated in FIG. 2, the tether member 110 can include a proximal aperture 122 and a distal aperture 124. In particular, the proximal member 118 can define the proximal aperture 122 and the distal member 120 can define the distal aperture 124. In some aspects, the proximal aperture 122 and the distal aperture 124 can be configured and arranged to receive at least a portion of the first and second coupling members 112, 114, respectively. In other words, the proximal and distal apertures 122, 124 can be configured to engage the first and second coupling members 112, 114, respectively, to engage the tether member 110 to the bone 102. Moreover, in some aspects, the proximal and distal apertures 122, 124 and the first and second coupling members 112, 114 can all be of a substantially similar size such that the first and second coupling members 112, 114 can be disposed through the proximal or distal apertures 122, 124. In other aspects, the proximal and distal apertures 122, 124 may include different sizes (e.g., different circumferences) such that the first and second coupling members 112, 114 uniquely engage the proximal and distal apertures 122, 124, respectively.

FIG. 3 illustrates one embodiment of the first and second coupling members 112, 114. As previously mentioned, in some aspects, the first and second coupling members 112, 114 can be substantially or completely identical. As such, the constituent elements of the first and second coupling members 112, 114 may be substantially or completely identical. Accordingly, although the following description relates to the first coupling member 112, in some embodiments, the constituent elements of the second coupling member 114 are the same or are substantially the same.

In some embodiments, the first coupling member 112 can be configured in a manner substantially similar to a coupling device, such as a screw (e.g., a cortical screw) or any other device that is capable of being disposed through the proximal or distal apertures 122, 124 to couple together the tether member 110 and the bone 102. For example, the first coupling member 112 can include a head 126 (e.g., a spherical head) that further includes a driving feature 128. In some aspects, the driving feature 128 can be configured to engage a device (e.g., a screw driver or like device) that an individual can use to apply a rotational force to the first coupling member 112 to drive the first coupling member 112 into the bone 102. Moreover, the first coupling member 112 may also include threading 130 that extends for at least a portion of a length of the first coupling member 112. Furthermore, the first coupling member 112 may also include a self-tapping and/or self-drilling tip 132 that can be used to improve the initial engagement of the first coupling member 112 and the bone 102.

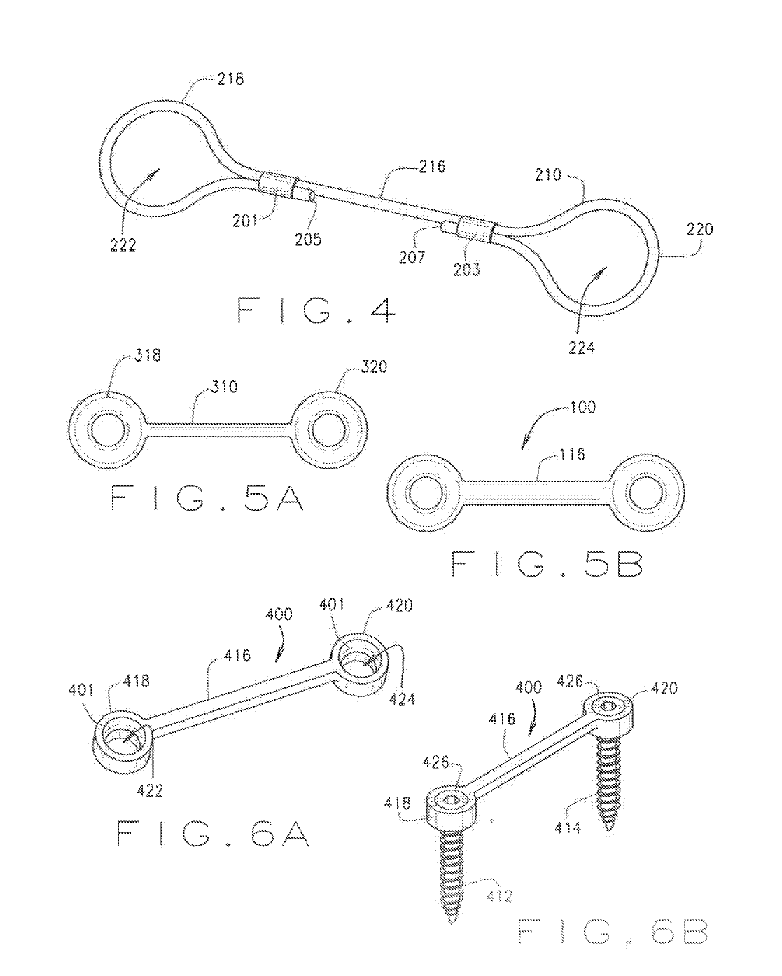

FIG. 4 illustrates another embodiment of a tether member, designated 210. In some aspects, the tether member 210 includes a central band 216, a proximal member 218, and a distal member 220. In some aspects, the tether member 210 can be fabricated from a cable, such as a mono- or a poly-filament cable, which exhibits a flexible nature. As such, during a procedure to affix the tether member 210 to a bone (not shown in FIG. 4), an appropriate length of cable can be cut from a stock. Thereafter, the cable can be actuated (e.g., bent over two fixed points, not shown in FIG. 4) to define the central band 216, the proximal member 218, the distal member 220, a proximal aperture 222, and a distal aperture 224 using a first attachment component 201 and a second attachment component 203. For example, after the individual using the tether member 210 determines a correct length of cable, the individual can obtain that length of cable from the stock. As such, the resulting length of cable has a first end 205 and a second end 207. Thereafter, the first and second ends 205, 207 can be respectively wrapped around a first and a second fixed point (not shown) and the first and second attachment components 201, 203 can be affixed to the tether member 210 to define the central band 216, the proximal member 218, the distal member 220, and the proximal and distal apertures 222, 224.

FIG. 5 illustrates yet another embodiment of a tether member, designated 310. In particular, the tether member 310 can be configured with different thicknesses. For example, the central band 316 can exhibit a reduced thickness relative to a thickness of the proximal and distal members 318, 320. Moreover, the central band 316 may also exhibit a reduced thickness relative to the central band 116 of tether member 110. In some aspects, some or all of the central band 316 and the proximal and distal members 318, 320 can exhibit a reduced thickness compared to the central band 116 and the proximal and distal members 118, 120 (reduced thickness not shown in FIG. 5).

FIGS. 6A and 6B illustrate another embodiment of the orthopedic apparatus, designated 400. In some embodiments, the orthopedic apparatus 400 can be fabricated using a machining process and be fabricated from a structurally strong but flexible material, such as certain metallic compounds or composite. Moreover, the orthopedic apparatus 400 can be formed with the central band 416 and the proximal and distal members 418, 420. In addition, the central band 416 can be configured as a flexible connection between the proximal and distal members 418, 420 for use in treating rotational bone deformities. Furthermore, the proximal and/or distal members 418, 420 can include a seating member 401 that is disposed circumferentially adjacent to the proximal and/or distal apertures 422, 424. In some aspects, the seating members 401 can be configured and arranged to engage a portion of the first and second coupling members 412, 414. For example, the seating members 401 can be configured and arranged to engage heads 426 of the first and second coupling members 412, 414 so that the heads 426 conform to the seating members 401.

As illustrated in FIGS. 1, 7A-7C, 8A, and 8B, regardless of configuration, in some embodiments, one or more orthopedic apparatuses 100 can be engaged to the bone 102 for the treatment of the rotational bone deformity (e.g., at least one orthopedic apparatus 100 coupled to a first and, laterally opposed, second side of the bone 102). For example, in some aspects, a first orthopedic apparatus 100 can be coupled to a lateral side of the bone 102 and a second orthopedic apparatus 100 can be coupled to a medial side of the bone 102 (not illustrated in the figures). In other aspects, more or less than two orthopedic apparatuses 100 can be coupled to the bone 102 to correct the rotational bone deformity.

In general, after diagnosis of a rotational bone deformity, the orthopedic apparatus 100 can be coupled to the bone 102 to promote torsional growth of the bone 102, which can result in substantial or complete correction of the rotational bone deformity. For example, the distal member 120 can be coupled to the fixed segment 106 of the bone 102 and the proximal member 118 can be coupled to the mobile segment 104 of the bone 102 using the first and second coupling members 112, 114. In addition, the tether member 110 can be positioned such that the coupling members 112, 114 or other elements of the orthopedic apparatus 100 do not contact or otherwise damage the growth plate 108. Moreover, as previously mentioned, another orthopedic apparatus 100 can be positioned in a similar manner on the opposite side of the bone 102 in a symmetrical or mirror-like configuration to provide the corrective torsional growth.

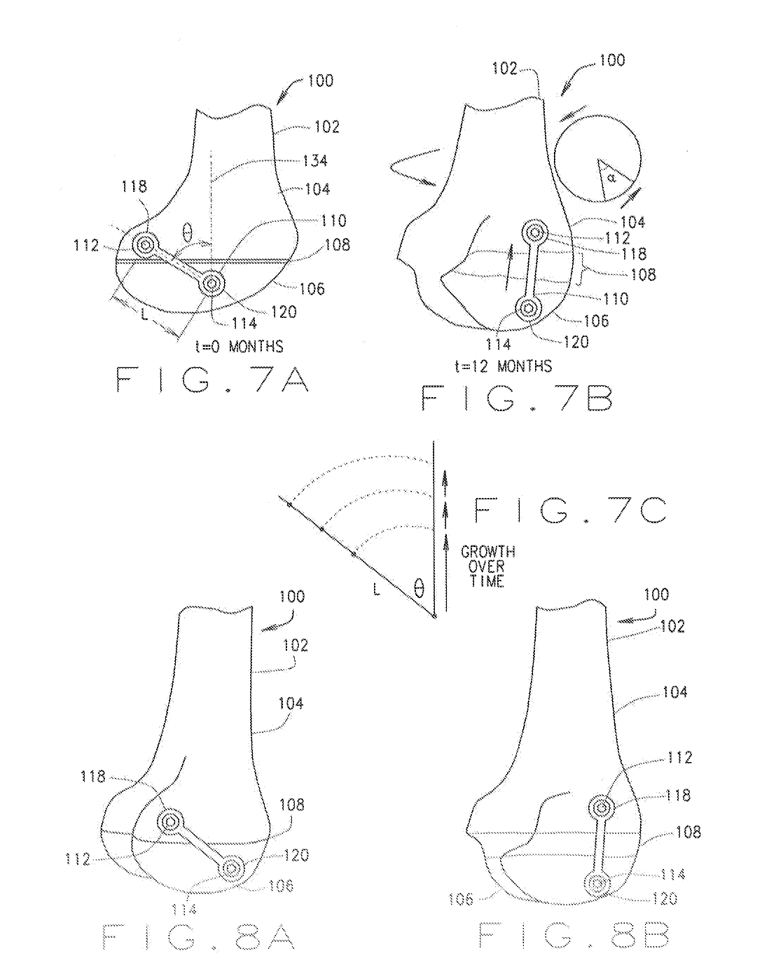

As illustrated in FIGS. 7A-7C, the positioning and length of the orthopedic apparatus 100 can be at least partially correlated with the extent of the rotational bone deformity of the bone 102. In particular, a length L of the tether member 110 can be selected at least partially based on a size of the bone 102 of the patient. Moreover, the tether member 110 can be coupled to the mobile and fixed segments 104, 106 of the bone 102 at a positioning angle θ relative to an anatomical axis 134 of the bone 102. As such, the positioning angle θ, length L, and tension associated with the orthopedic apparatus 100 can promote torsional growth of the growth plate 108, thereby correcting the deformation, as illustrated in FIGS. 7B and 8B. Accordingly, the orthopedic apparatus 100 can function as a tethering cable such that the orthopedic apparatuses 100 placed on opposite sides of the bone 102 produce equal and opposite forces on the growth plate 108 to force rotation of the bone 102.

In addition, the tether member 100 can be coupled to the mobile and fixed segments 104, 106 of the bone 102 such that some or all of the slack of the tether member 110 is eliminated. The elimination of some or all of the slack in the tether member 110 can result in substantially sufficient tension of the tether member 110 to affect correction of the bone deformity. Moreover, the tension of the tether member 110 can be maintained by growth of the bone 102 such that no springs or other types of biasing members are required for use of the orthopedic apparatus 100. In other embodiments, a biasing member (not shown) may be used to aid in providing sufficient tension in the tether member 110.

During growth of the bone 102, the tethering effect and the positioning of the orthopedic apparatuses 100 on opposing sides of the bone 102 (not shown in the figures) induce torsion forces on the nascent bone cells of the growth plate 108. For example, as the mobile segment 104 of the bone 102 moves away from the growth plate 108, the orthopedic apparatus 100 induces torsion forces on the mobile segment 104 and the growth plate 108 until the orthopedic apparatus 100 reaches a substantially or completely vertical position (e.g., the angle θ becomes closer to zero degrees) such that the orthopedic apparatus 100 is positioned parallel or substantially parallel to the anatomical axis 134. In other words, after being positioned, the tether member 110 can freely rotate about an axis 136 of the first and second coupling members 112, 114 (as shown in FIG. 2) while remaining in contact with the bone 102 and maintaining tension.

In addition, an anteversion-retroversion rotation correction angle α (as shown in FIG. 7B) can be managed by the length L and the positioning angle θ. For example, a longer tether member 110 will enable more growth at the growth plate 108 because it will take a greater amount of time for the orthopedic apparatus 100 to reach the vertical position. Moreover, a combination of length L and positioning angle θ allows for control over treatment time and can maximize de-rotation effect, as illustrated in FIG. 7C. In addition, geometry of the particular bone 102 can also impact the treatment time and the anteversion-retroversion rotation correction angle α.

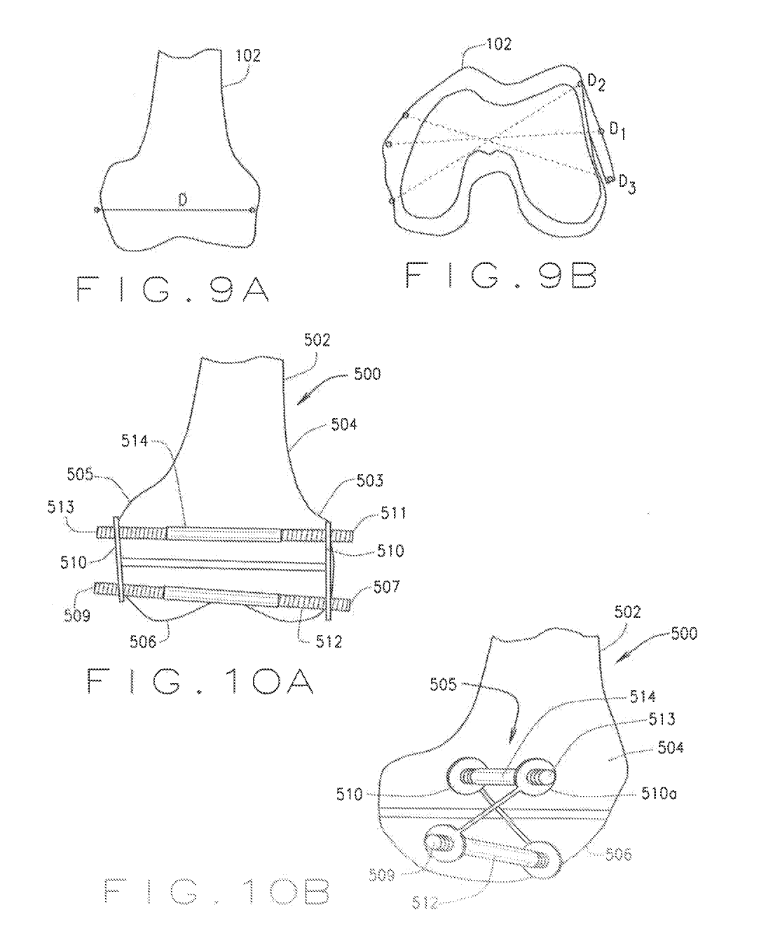

As illustrated in FIGS. 9A and 9B, a femur condylar distance D can be used as a reference for the selection of an appropriately sized tether member 110. In particular, the femur condylar distance D can serve as a symmetric anchorage distance under the growth plate 108 for each of the orthopedic apparatuses 100 (not shown in FIGS. 9A and 9B). Moreover, femur condylar distances D1, D2, D3 illustrate multiple potential positions for the orthopedic apparatuses 100.

Once in a vertical or substantially vertical position, the orthopedic apparatus 100 can function as a growth plate 108 hemiepiphysiodesis device (e.g. staple) to prevent further growth. As such, to prevent growth arrest, the orthopedic apparatus 100 can be removed before reaching the vertical position or just upon reaching the vertical position. If further correction is necessary after the orthopedic apparatus 100 reaches the vertical position, additional orthopedic apparatuses 100 can be coupled to the mobile and fixed segments 104, 106 of the bone 102 and the process can be repeated.

In another application, if the goal of the procedure is to stop growth (physiodesis), the apparatus can be implanted in the vertical position on one or both sides of the bone 102.

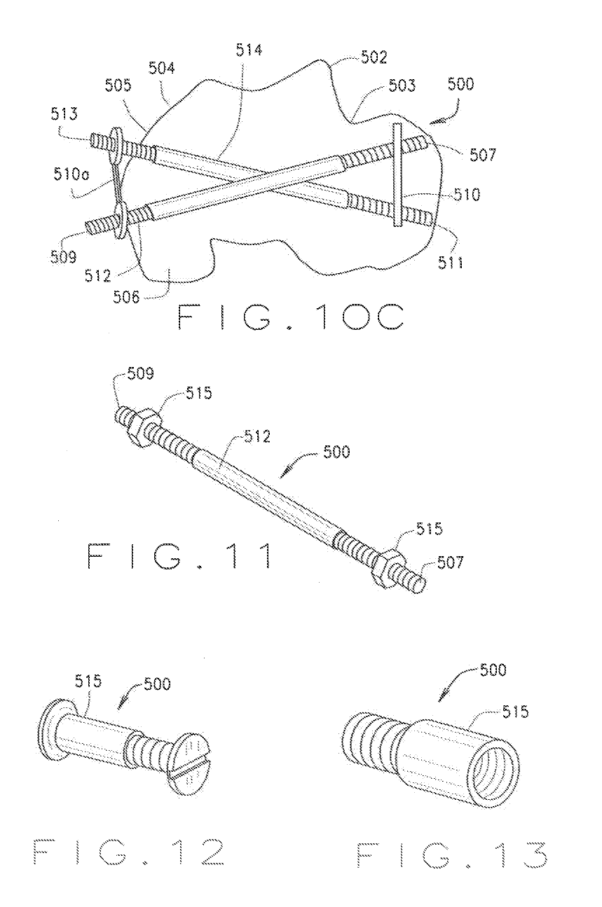

FIGS. 10A-10C illustrate another embodiment of the orthopedic apparatus, designated 500. Similar to other embodiments, a first tether member 510 can be coupled to a medial side 503 of the bone 502 and a second tether member 510a can be coupled to a lateral side 505 of the bone 502. In some embodiments, the first and second tether members 510, 510a can be coupled to the mobile and fixed segments 504, 506 of the bone 502 using first and second coupling members 512, 514 that extend through the entire distance of the bone 502. In particular, the first coupling member 512 can be inserted into the fixed segment 506 such that a first end 507 of the first coupling member 512 can extend from the medial side 503 of the bone 502 and a second end 509 of the first coupling member 512 can extend from the lateral side 505 of the bone 502. Similarly, the second coupling member 514 can be inserted into the mobile segment 504 such that a first end 511 of the second coupling member 514 can extend from the medial side 503 of the bone 502 and a second end 513 of the second coupling member 514 can extend from the lateral side 505 of the bone 502. After positioning of the first and second coupling members 512, 514 through the bone 502, the tether members 510, 510a can be positioned such that the first and second ends 507, 509 of the first coupling member 512 extend through the proximal apertures of the first and second tether members 510, 510a, respectively. Similarly, the tether members 510, 510a may also be positioned such that the first and second ends 511, 513 of the second coupling member 514 extend through the distal apertures (not shown) of the first and second tether members 510, 510a, respectively.

As illustrated in FIG. 11, the first and second coupling members 512, and the second coupling member 514 (not shown) can be configured to engage one or more securing members 515 to secure in place the first and second tether members 510, 510a shown in FIGS. 10A-10C. In some embodiments, a portion of the first and second coupling members 512, 514 adjacent to the first ends 507, 511 and the second ends 509, 513 can exhibit a threaded configuration that is capable of engaging the securing members 515. For example, the securing members 515 can be configured as nuts that engage the first and second coupling members 512, 514 to secure the tether members 510, 510a. As illustrated in FIGS. 12 and 13, the securing members 515 used with orthopedic apparatus 500 can be configured in alternative manners to reduce the possible excessive protrusion of the threads from the bone 502. For example, some or all of the securing members 515 can be configured as binding posts (FIG. 12) or barrel extensions (FIG. 13). In other embodiments, the securing members 515 can exhibit any other configuration that can engage the first and second coupling members 514, 516 to retain in place the first and second tether members 510, 510a.

Overall, some embodiments of the orthopedic apparatus 100 can provide benefits compared to conventional systems that are used to correct rotational bone deformities. For example, at least one conventional system uses plates or plate-like apparatuses to limit growth of the bone 102 and induce torsion. However, the use of rigid plates presents a significant problem in regards to bone geometry, which is overcome by at least some embodiments of the orthopedic apparatus 100. In particular, the flexible nature of the orthopedic apparatus 100 provides the capability to adjust to personalized bone geometry that would not be capable with rigid plates. In addition, the symmetrical positioning of at least two orthopedic apparatuses 100 provides equal, but opposite torsional forces, which is not seen with rigid plates. Moreover, rigid plates have an increased risk of jamming due to a misalignment of the plates, which can lead to growth arrest and/or induce unwanted deformities. Some or all embodiments of the orthopedic apparatus 100 do not present a significant risk of jamming. All together, the orthopedic apparatus 100 does not suffer from the significant drawbacks exhibited by some conventional systems.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. An orthopedic apparatus comprising:
a tether member comprising:
a central band being disposed between a distal member and a proximal member;
a proximal aperture being defined by the proximal member;
a distal aperture being defined by the distal member; and
wherein the distal member is configured to be engaged to a first segment of a bone and the proximal member is configured to be engaged to a second segment of the bone; and
a plurality of coupling members being, each of the plurality of coupling members configured to engage the distal member to the first segment of the bone and the proximal member to the second segment of the bone.

2. The orthopedic apparatus of claim 1, wherein the plurality of coupling members comprises a first coupling member and a second coupling member.

3. The orthopedic apparatus of claim 1, wherein at least one of the plurality of coupling members comprises a cortical screw.

4. The orthopedic apparatus of claim 1, wherein the central band, the distal member, and the proximal member each comprise a respective thickness, and further wherein the respective thickness of the central band is less than the respective thicknesses of the distal member and the proximal member.

5. The orthopedic apparatus of claim 1, wherein the first segment of the bone and the second segment of the bone are on opposing sides of a growth plate of the bone.

6. The orthopedic apparatus of claim 1, wherein the bone comprises an anatomical axis, and further wherein the tether member is coupled to the bone at an angle relative to the anatomical axis.

7. The orthopedic apparatus of claim 1 and further comprising at least one seating member defined by the distal member and the proximal member.

8. The orthopedic apparatus of claim 1, wherein the central band is fabricated from a flexible material.

9. A method of correcting rotational deformities of a bone, the bone including a fixed segment separated from a mobile segment by a growth plate and a first side of the bone laterally opposing a second side of the bone, the method comprising:
coupling a first portion of a first tether member to the fixed segment of bone on the first side of the bone using a first coupling member;
coupling a second portion of the first tether member to the mobile segment of the bone on the first side of the bone using a second coupling member, wherein the first tether member is coupled to the fixed segment of the bone and the mobile segment of the bone such that a central band of the first tether member is under tension;
coupling a first portion of a second tether member to the fixed segment of bone on the second side of the bone using a third coupling member; and
coupling a second portion of the second tether member to the mobile segment of the bone on the second side of the bone using a fourth coupling member, wherein the second tether member is coupled to the fixed segment of the bone and the mobile segment of the bone such that a central band of the second tether member is under tension.

10. The method of claim 9 and further comprising uncoupling the first tether member and the second tether member from the bone.

11. The method of claim 9, wherein the first tether member and the second tether member are coupled to the first side of the bone and the second side of the bone in a symmetrical manner.

12. The method of claim 9, wherein the first coupling member, the second coupling member, the third coupling member, and the fourth coupling member are disposed in the bone such that the first coupling member, the second coupling member, the third coupling member, and the fourth coupling member are oriented substantially parallel to the growth plate or are substantially angled away from the growth plate.

13. The method of claim 9, wherein the bone comprises an anatomical axis, and wherein the first tether member and the second tether member are coupled to the bone at an angle relative to the anatomical axis.

14. The method of claim 13, wherein the first tether member and the second tether member are coupled to the bone such that as growth occurs at the growth plate, the angle relative to the anatomical axis becomes closer to zero degrees.

15. The method of claim 9, wherein at least one of the first coupling member, the second coupling member, the third coupling member, and the fourth coupling member is a cortical screw.

16. A method of correcting rotational deformities of a bone, the bone including a fixed segment separated from a mobile segment by a growth plate and a first side of the bone laterally opposing a second side of the bone, the method comprising:
positioning a first coupling member through the fixed segment of the bone, the first coupling member including a first end opposing a second end, wherein the first coupling member is positioned through the fixed segment of the bone such that the first end of the first coupling member extends from the first side of the bone and the second end of the first coupling member extends from the second side of the bone;
positioning a second coupling member through the mobile segment of the bone, the second coupling member including a first end opposing a second end, wherein the second coupling member is positioned through the mobile segment of the bone such that the first end of the second coupling member extends from the first side of the bone and the second end of the second coupling member extends from the second side of the bone;
positioning a first tether member on the first side of the bone such that the first end of the first coupling member and the first end of the second coupling member engage the first tether member; and
positioning a second tether member on the second side of the bone such that the second end of the first coupling member and the second end of the second coupling member engage the second tether member.

17. The method of claim 16, further comprising engaging at least one securing member to at least one of the first end and second end of the first coupling member.

18. The method of claim 17, further comprising engage at least one securing member to at least one of the first end and the second end of the second coupling member.

19. The method of claim 16, wherein the bone comprises an anatomical axis, and wherein the first tether member and the second tether member are positioned on the first side and second side of the bone at an angle relative to the anatomical axis.

20. The method of claim 19, wherein the first tether member and the second tether member are positioned on the first side and second side of the bone such that as growth occurs at the growth plate, the angle relative to the anatomical axis becomes closer to zero degrees.
